(19) United States
(12) Patent Application Publication
(10) Pub. No.: US 2002/0009501 A1
(43) Pub. Date: Jan. 24, 2002

(54) Preparation of cartilage extracts using organic solvents

(76) Inventors: Eric Dupont, Saint Nicolas (CA); Yves Lachance, Saint Nicolas (CA); Denis Lessard, Levis (CA); Serge Auger, Saint Lambert (CA)

Correspondence Address: RANDALL C BROWN, AKIN GUMP STRAUSS HAUER & FELD, P O BOX 688, DALLAS, TX, 75313

(21) Appl. No.: 09/776,765

(22) Filed: Feb. 5, 2001

Related U.S. Application Data
(63) Continuation-in-part of application No. 09/751,111, filed on Dec. 29, 2000.
Application No. 09/751,111 is a continuation of application No. 09/122,481, filed on Jul. 23, 1998, now Pat. No. 6,168,807.

(51) Int. Cl. A61K 35/34

(52) U.S. Cl. 424/548

(57) ABSTRACT

This invention relates to a process by which organic solvent-containing solutions are used in lieu of pure water for the preparation of cartilage extracts and fractions thereof. Different organic solvents have been tested for the preparation of extracts containing biologically active components. Amongst the tested solvents, trimethylamine 40% (in water) was selected as a good alternative solvent to pure water, particularly in recovering an anti-proliferative activity against HUVECs.

### CROSS-REFERENCES TO RELATED APPLICATIONS

This application is a Continuation-in-Part of U.S. patent application Ser. No. 09/751,111 which is a Continuation of U.S. patent application Ser. No. 09/122,481, the entire disclosures of which are hereby incorporated by reference.

### FIELD OF THE INVENTION

This invention relates to a process for extracting biologically active components from cartilage tissue. Particularly, the process makes use of organic solvents combined or not with water. Organic solvents may be used to selectively extract some active components at the expense of others. Therefore, extracts enriched in some activities, either anti-metalloprotease (namely anti-MMP-2) activity, or anti-proliferative activity against HUVECs are obtained.

### BACKGROUND OF THE INVENTION

Processes for the preparation of shark cartilage extracts and the extracts themselves are disclosed in International Publications WO 95/32722, WO 96/23512 and WO 97/16197. Liquid extracts of shark cartilage have been tested in various assays for antiangiogenic, anticollagenolytic, direct anti-tumor proliferating and anti-inflammatory activities.

WO 95/32722 discloses a process for obtaining a shark cartilage extract having antiangiogenic, in vitro direct anti-tumor proliferating and in vivo anti-tumor activities. That process comprises the steps of blending shark cartilage tissue and reducing the same to a particle size of about 500 μm in water; extracting active components into the water; and fractionating the extracts so obtained in order to recover molecules having molecular weights less than about 500 kDa (0-500 fraction). The liquid cartilage extract was concentrated on a membrane having a nominal porosity of about 1 kDa to form a concentrated liquid extract comprising molecules having molecular weights less than about 500 kDa. The extract was enriched in molecules having molecular weights between about 1-500 kDa. The 0-500 fraction was further fractionated to form a plurality of extracts containing anti-tumor proliferating molecules having molecular weights extending from about 1 to 120 kDa. The WO 95/32722 Publication does not disclose the specific recovery of components having molecular weights less than about 1 kDa. It also does not disclose a process of obtaining a cartilage extract or fractions thereof in organic solvent-containing solutions.

International Publication No. WO 96/23512 discloses a process for extracting biologically active components from any source of cartilage in aqueous solutions. Further, this publication discloses other biological activities associated with the liquid shark cartilage, namely anticollagenolytic and anti-inflammatory activities. The WO 96/23512 Publication does not disclose the recovery of components having molecular weights less than about 1 kDa nor any process making use of organic solvent-containing solutions.

International Publication No. WO 97/16197 discloses a process for the recovery of an aqueous extract enriched in molecules having molecular weights between about 0.1 to 500 kDa. Although that process may recover components having molecular weights of less than about 1 kDa, it does not provide for any recovery of specific low molecular weight components. No component in an isolated or purified form is disclosed.

It is generally accepted in the art that matrix metalloproteases are involved in the processes of neovascularization, promoting the growth of primary tumors and in the formation of metastases. Accordingly, compounds or agents exhibiting antiangiogenic and/or anti-matrix metalloprotease activities are believed to be useful for at least one of inhibiting neovascularization, inhibiting growth in tumors, inhibiting metastatic invasion of cells, inhibiting formation of metastases and treating angiogenesis related diseases.

Given the interest in components obtained from shark cartilage, there exists the need for improved processes for their preparation and for the isolation and purification of other components not previously known to possess biological activity.

### SUMMARY OF THE INVENTION

The present invention seeks to provide improved processes for the preparation of extracts obtained from cartilage.

In one aspect, the present invention provides a process wherein a variety of conditions are used for the preparation of cartilage extracts and fractions thereof containing biologically active components. In one embodiment, the invention provides a process for the preparation of shark cartilage extracts having components possessing at least an anti-MMP a, anti-PPE and anti-proliferative in HUVECs activities. This process makes use of organic solvents.

In another aspect, the present invention provides a process by which the 0-500 molecular weight fraction of biologically active components derived from a cartilage liquid extract is separated into two separate fractions wherein the first fraction comprises components having molecular weights less than about 1 kDa (0-1 fraction) and the second fraction comprises components having molecular weights between about 1 to 500 kDa (1-500 fraction).

In order to minimize the formation of component aggregates, to improve the dissolution and the maintenance of a, stable, soluble form, sucrose or one or more other suitable stabilizers such as dextran, Ficoll™, fructose, gelatin, glucose, glycine, inositol, lactose, mannitol and sorbitol can be added in a sufficient stabilizing amount to any of the 0-500, 0-1 and 1-500 fractions, or can be used in any step of the manufacturing process. As used herein in reference to fractions, solutions or extracts, the phrase “containing 1% w/v sucrose” refers to a respective fraction, solution or extract containing about 1% w/v sucrose. Biologically active components in the 0-500, 0-1 and 1-500 fractions possess anti-MMP, anti-elastase and antiangiogenic activities. The solvents and their concentration in water influence the nature of the extracts.

In another aspect, the present invention provides a shark cartilage derived component having a molecular weight of about 244 amu (atomic mass unit), herein termed [symbol] -986, possessing at least one of anti-MMP and anti-tumor activities. The process and materials used for the purification of the [symbol] -986 reveal some physico-chemical characteristics of the latter, which are responsible for the partitioning of this component in different solvent phases and chromatographic systems. The present invention also provides a process for the isolation and purification of the [symbol] -986 component or of an equivalent component obtained from any source of cartilage.

Yet another aspect of the invention provides a purified biologically active compound derived from any source of cartilage which corresponds to the compound having a molecular weight of about 244 amu isolated from shark cartilage and possessing anti-MMP activity.

Still another aspect of the invention provides a method of inhibiting a MMP enzyme, which method comprises the step of contacting a substrate cleavable by said enzyme with an effective amount of one or more cartilage extracts or fractions derived therefrom.

Still other aspects of the invention provide methods of inhibiting neovascularization and the formation of metastases, which methods comprise the step of contacting a target tissue with an effective amount of a cartilage derived extract, solution, homogenate, suspension, fraction such as the 0-500 fraction, the 0-1 fraction, the 1-500 fraction or the same fractions containing 1% w/v sucrose.

### BRIEF DESCRIPTION OF THE DRAWINGS

The following figures are part of the present specification and are included to further demonstrate certain aspects of the invention. The invention may be better understood by reference to one or more of these figures in combination with the detailed description of the specific embodiments presented herein.

FIG. 1 represents the concentration of different shark cartilage extracts (μg/mL) causing 50% inhibition in the PPE enzymatic assay. The IC₅₀ is plotted against increasing concentrations of solvent.

FIG. 2 represents the concentration of different shark cartilage extracts (μg/mL) causing 50% inhibition in the MMP-2 enzymatic assay. The IC₅₀ is plotted against increasing concentrations of solvent.

FIG. 3 represents the concentration of different shark cartilage extracts (μg/mL) causing 50% inhibition in the HUVEC enzymatic assay. The IC₅₀ is plotted against increasing concentrations of solvent.

FIG. 4 represents the relationship between HPSEC length ratio versus the protein content of the extracts obtained in different solvents.

FIG. 5 represents the relationship between HPSEC vector angle versus the protein content of the extracts obtained in different solvents.

### DETAILED DESCRIPTION OF THE INVENTION

Biological Assays

The biological properties of shark cartilage extracts, of fractions derived therefrom and of the component [symbol] -986 were determined by using at least one of the following assays:

Gelatinase Inhibition Assay (GIA): an assay for evaluating anti-MMP activity;

Embryonic Vascularisation Test (EVT): an assay for evaluating antiangiogenic activity; and

Lewis Lung Carcinoma metastatic mouse model (LLC): an assay for evaluating anti-tumor activity:

GIA

The GIA was performed using a commercial kit (Boehringer Mannheim). The GIA is used to determine the ability of components in the cartilage derived extracts, or fractions thereof or of the [symbol] -986 component to inhibit the activity of the gelatinase A enzyme (MMP-2).

Briefly, the GIA was performed as follows. A biotin-labeled gelatine substrate was incubated with gelatinase A in the absence or the presence of a liquid cartilage extract or its derivatives. Subsequently, the reaction mix was loaded onto a streptavidin-coated microtiter plate. The biotin-labeled gelatine was bound to the streptavidin-coated microtiter via its free biotin residues. If the substrate, gelatine, was not spliced by gelatinase, a streptavidin-peroxidase (POD) conjugate bound to the gelatinase-biotin-complex. The POD then converted an added ABTS substrate to a green end product, which was measured at 405 nm. However, if the biotin-labeled gelatine was spliced by gelatinase, only small fragments of gelatine were formed. These fragments, after attachment to a microtiter plate, did not possess the ability to bind the streptavidin-POD conjugate; and therefore, no color reaction occurred.

High gelatinase activity thereby yields low signals, and a low gelatinase activity in turn (e.g. by addition of an inhibitor) causes high signals. The activity sought for the components in a cartilage derived extract, or fractions derived therefrom, may be an inhibitory activity towards gelatinase or an antagonist activity which competes with the interaction between gelatinase and its gelatine substrate (e.g. the antagonist components bind gelatine).

EVT

The Embryonic Vascularization Test (EVT) was performed to determine the ability of components in the shark cartilage liquid extracts, or fractions derived therefrom, to inhibit the formation of new blood vessels (antiangiogenic activity).

The normal development of a chick embryo involves the formation of an external vascular system located in the vitelline membrane which carries nutrients from the vitellus (yolk) to the developing embryo. When placed onto the vitelline membrane, antiangiogenic substances can inhibit the blood vessel formation that occurs in the vitelline membrane.

Methylcellulose discs (an inert solid and transparent matrix) containing different quantities of components from shark cartilage derived liquid extracts, or fractions derived therefrom or appropriate controls were placed on the external border of the vascular perimeter of the vitelline membrane, where the angiogenic process occurs. Positive controls consisted of methylcellulose discs containing 1.5 mg/ml of 2-Methoxyestradiol. Control and sample-containing discs were placed onto the vitelline membrane of 3 day-old embryos. At this point, only beginnings of the main blood vessels are invading the vitellus. Methylcellulose discs containing a negative control or an amount of components from shark cartilage derived liquid extract or fractions derived therefrom were always placed on the vitelline membrane of the same embryo concurrently. Both discs were arranged in a symmetric fashion with respect to the cephalo-caudal axis of the embryo in order to minimize inter-individual variations when comparing the efficacy of said components to that of negative controls. Vascularization was assessed 24 hours after disc deposition, and results were expressed as the percent of embryos in which blood vessel formation was affected. The blood vessel formation was considered affected when its growing path was either deviated, or diminished or when there was no growth observed beyond the disc as compared to the negative control.

LLC model

The Lewis Lung Carcinoma mouse model (LLC) was used to determine the ability of components of shark cartilage liquid extracts, or of fractions derived therefrom or of the [symbol] -986, to inhibit the formation of metastases within lung.

Cell Culture:

The Lewis lung carcinoma clone M27, with a high metastatic potential to the lung, was established by Dr P. Brodt (Brodt P, Cancer Res., 46: 2442, 1986). This model is well established and is known for its predictive correlation between in vitro and in vivo activity. Cells were maintained in RPMI-1640 medium supplemented with 10% fetal bovine serum and 1% penicillin-streptomycin, under 5% CO₂ and were passaged twice a week. Stocks of the cells were generated and stored as early passages. All experiments were carried out using the same passage. For tumor induction, M27 cells were grown at 70% confluence in complete medium and then collected using trypsin-EDTA solution (0.05% trypsin, 0.53 mM EDTA-4Na in HBSS without Ca++ or Mg++). Cells were then centrifuged, washed and resuspended (1×10⁶ LLC cells per 200 μl of PBS Ca++ and Mg++ free). Viability was examined by tryptan blue staining and only flasks in which the viability was superior to 95% were used for inoculation.

Tumor Induction:

C57BL/10 female mice (15 to 20 g) (Charles River Inc.) were used to induce the Lewis lung carcinoma tumors. After one week of incubation, LLC cells were transplanted subcutaneously (5×10⁵ viable cells per 100 μl) in the axillary region of the right flank at day 0. All animals were inoculated at the same site. Tumor growth was monitored every day using calipers. The relative tumor volume was calculated using the formula: length (cm)×[width (cm)]²/2 where the length corresponds to the longest axis and the width corresponds to the perpendicular shortest axis of the tumor. When the primary tumor reaches a size of 0.5-1.0 cm³ (day 10 post-inoculation), mice bearing primary tumors of approximately identical size were randomly assigned to specific experimental groups of 15 animals each and labeled by numbers using the ear punching method. Surgery was performed under sterile conditions. Following a small skin incision (0.5-1 cm), the tumor was carefully separated from the surrounding healthy tissues. LLC cells (at early stage of growth) form a well localized tumor and separation was easy to achieve without any significant damage to normal tissues. Stereoscopic examination revealed the absence of any macroscopic residual tumor at the site of tumor inoculation and tumor regrowth was not observed under our conditions. Following removal, tumor was weighted and the wound was closed with surgical stainless steel clips and disinfected with providone-iodine.

Efficacy Study Experimental Design:

Treatment with different test samples (components derived from shark cartilage liquid extracts, fractions derived therefrom or [symbol] -986) started the day following tumor removal (day 11 post-inoculation). Saline or the cartilage-derived products were given daily for two weeks by oral gavage. Oral gavage (0.5 ml) was performed using a 22G curved needle. As previous experiments had shown that a period of approximately two weeks after removal of the primary tumor was sufficient to obtain an average of 30 to 50 nodules on the lung surface, animals were sacrificed in a CO₂ chamber two weeks later. Following autopsy, both lungs were removed, weighed and fixed in 10% Bouin's fixative. Lung surface metastases were counted using a stereomicroscope (4×).

Measurement of Body Weight:

Body weight was monitored every second or third day until sacrifice.

Processes for Preparing Cartilage Extracts

Extraction of Active Components from Shark Cartilage Using Organic Solvent-containing Solutions

The present invention provides a method of preparing a cartilage extract and of obtaining, isolating or purifying therefrom biologically active components therein, wherein at least a portion of the biologically active component is not of a protein nature. However, chaotropic agents which are useful for extracting protein-containing components may be used in the process of the present invention.

As used herein, the term “organic solvent-containing solution” refers to a solution or mixture comprising at least a portion of organic solvent. The organic solvent-containing solution can comprise one or more organic solvents and can contain water. An organic solvent or combination of organic solvents used herein is preferably polar. In one embodiment, at least one of methanol and ethanol can be used for the preparation of shark cartilage liquid extracts. Other organic solvents such as acetonitrile, propanol, isopropanol and acetone are suitable polar solvents that can be used. The organic solvent can include one or more halogenated, ether, protic, aprotic, polar, apolar, basic, acidic, hydrophobic, and hydrophilic solvents.

Suitable halogenated solvents include: chloroform, dibromomethane, butyl chloride, dichloromethane.

Suitable ether solvents include: dimethoxymethane, tetrahydrofuran, diethyl ether, ethylene glycol dimethyl ether, ethylene glycol diethyl ether, diethylene glycol diethyl ether, triethylene glycol dimethyl ether, t-butyl ethyl ether, or t-butyl methyl ether.

Suitable protic solvents may include, by way of example and without limitation, methanol (MeOH), ethanol (EtOH), 2-nitroethanol, 2-fluoroethanol, 2,2,2-trifluoroethanol, ethylene glycol, 1-propanol, 2-propanol (ISO), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, anisole, benzyl alcohol, phenol, or glycerol.

Suitable aprotic solvents may include, by way of example and without limitation, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile (ACN), dimethyl sulfoxide (DMSO), propionitrile, ethyl formate, methyl acetate, acetone, ethyl methyl ketone, ethyl acetate, sulfolane, N,N-dimethylpropionamide, tetra methylurea, nitromethane, nitrobenzene, or hexamethylphosphoramide.

Suitable basic solvents or solutions include: 2-, 3-, or 4-picoline, pyrrole, pyrrolidine, ammonium hydroxyde (NH₄OH), trimethyl amine (TMA), morpholine, pyridine, or piperidine.

Suitable acidic solvents or solutions include trifluoroacetic acid (TFA), acetic acid, proprionic acid or formic acid.

Suitable hydrocarbon solvents include: benzene, cyclohexane, pentane, hexane, toluene, cycloheptane, methylcyclohexane, heptane, ethylbenzene, octane, indane, nonane, or naphthalene.

The organic solvent-containing solution can comprise combinations of organic solvents and/or combinations of organic solvents and water. Suitable protic solvent combinations with water can include, by way of example and without limitation, water-methanol, water-propanol, water-isopropanol, water-butanol. Suitable aprotic solvent combinations with or without water can include, by way of example and without limitation, water-acetonitrile, water-dimethylsulfoxide, methanol-acetonitrile, methanol-dimethylsulfoxide, ethanol-acetonitrile, and ethanol-dimethylsulfoxide.

The amount of organic solvent present in the invention can vary according to the nature or physical properties of a component to be extracted from cartilage. In general, the organic solvent-containing solution will contain about 0.1, 1-100% v/v, about 40-80% v/v, at least 1% v/v, at least 10% v/v, at least 25% v/v, at least 50% v/v, at least 90% v/v or at least 99% v/v organic solvent with respect to the total solution volume. The amount of basic or acidic solvents can vary from about 0.1 to about 50% depending on the pKa of the solvents. The more extreme pKa values are, the lesser are the concentrations of basic or acidic solvents, to avoid destruction or denaturation of the biological components.

Accordingly, the present invention provides a process for the preparation of extracts of shark cartilage comprising the steps of:

a) treating shark cartilage material with a quantity of organic solvent-containing solution to form a first mixture comprising soluble components of shark cartilage;

b) separating said first mixture to form a first liquid extract comprising said soluble components and a first mass of solids; and

c) removing the organic solvent from said first liquid extract.

The process can further comprise the steps of:

d) removing a sufficient amount of liquid from said first liquid extract to form a substantially dry second mass of solids;

e) adding water to said second mass of solids to form a second mixture; and

f) separating said second mixture to form a first final liquid extract and a third mass of solids.

The first mass of solids containing the shark cartilage material can be extracted an additional one or more times with an organic solvent-containing solution, or water in place of the organic solvent containing solution, according to the steps a) through c) described above to form second and third or further final liquid extracts containing at least residual amounts of soluble components of shark cartilage.

The separation of solids and liquid in step b) can be conducted according to any of a number of methods known to those of skill in the art including, by way of example and without limitation, centrifugation, filtration, diafiltration, ultrafiltration, microfiltration, and settling of solids and removal of supernatant.

The removal of organic solvent, as indicated in step c), can be done according to any of a number of methods known to those of skill in the art including, by way of example and without limitation, evaporation, lyophilization, distillation, desiccation, addition of organic solvent absorbent, liquid/liquid extraction and rotovapping.

The shark material used herein will be a solid and can be, for example, a powder, granulate, rod, or particle. Prior to or during step a), the shark material can be homogenized. As used herein, the terms “homogenize”, “homogenizing” and “homogenization” refer to a process of increasing the efficiency of extraction of desired components from cartilage material by either: a) increasing the total or specific surface area of the cartilage material, or b) facilitating the release of desired components from the cartilage material. The homogenization can be conducted by one or more of chemical means, physical means and combinations thereof.

Chemical means for homogenizing the cartilage material will include one or more chemical agents that swell the cartilage material, disrupt or lyse cells or extracellular matrix in the cartilage material, and/or increase the porosity of the cartilage material. Exemplary non-limiting examples of such chemical agents include detergents, surfactants, ionic agents, nonionic agents, reducing agents, chelators, glycosylating agents, chaotropic agents, urea, guanidine, phospholipids, glycolipids, dithiothreitol, β-mercaptoethanol, sodium lauryl sulfate, triton solution and other such agents known to those of skill in the art or disclosed in “A Guide to the Properties and Uses of Detergents in Biology and Biochemistry” by Judith Neugebauer (Calbiochem-Novabiochem Corporation, 1988) the disclosure of which is hereby incorporated by reference.

Physical means for homogenizing the cartilage material will generally result in reducing the average particle size of the shark material thereby increasing its specific surface area. The particle size reduction can be done by any one or more of the following exemplary methods including pulverization, micronization, milling, grinding, chopping, blending under high speed and other methods known to those of skill in the art of particle size reduction.

The extraction solutions can contain extraction enhancing agents which enhance the extraction of components from cartilage. These extraction enhancing agents can include inorganic or organic acids, inorganic or organic bases, polymers, buffers, salts and other similar agents known to those of skill in the art.

According to one embodiment, the extraction of low molecular weight materials from cartilage was done by:

a) treating homogenized shark cartilage material (1 kg) with methanol (1 kg) to form a first mixture comprising soluble components of shark cartilage;

b) centrifuging said first mixture to form a first liquid extract comprising said soluble components and a first mass of solids;

c) evaporating the methanol from said first liquid extract;

d) evaporating a sufficient amount of liquid from said first liquid extract to form a substantially dry second mass of solids;

e) adding water (1 kg) to said second mass of solids to form a second mixture; and

f) centrifuging said second mixture to form a first final liquid extract and a third mass of solids.

Steps c) and d) above can be optionally combined to go directly from the first liquid extract to the second mass of solids.

All the liquid extracts resulting from extractions and reiterated extractions of the shark cartilage, from the above steps, were analyzed for their dry weight content and protein concentrations (as determined by a standard Bradford protein assay) as an indication of the recovery of soluble components. The anti-MMP activity was also evaluated. The GIA was conducted on 40 μl of 20× concentrated samples. The results are summarized in Table 1.

TABLE 1

| | | Protein | |
|---|---|---|---|
| | Dry weights | concentration | |
| Fractions tested | (mg/ml) | (μg/ml) | GIA (% inhibition) |
| CTRL-S1 | 21.9 | 2133.8 | 72 |
| CTRL-S2 | 12.1 | 1016.3 | 42 |
| CTRL-S3 | 6.2 | 758.6 | 47 |
| SU-MET-S1 | 14.3 | 54.8 | 52 |
| SU-MET-S2 | 6.1 | 28.6 | 13 |
| SU-MET-S3 | 3.4 | 48.5 | 0 |
| SU-ETH-S1 | 5.5 | 30.8 | 16 |
| SU-ETH-S2 | 7.1 | 79.5 | 4 |
| SU-ETH-S3 | 2.9 | 63.7 | 0 |

As used in Table 1, “CTRL” (control sample) indicates a final liquid extract obtained when using purified water as the extraction solvent. The term “SU-MET” indicates a final liquid extract obtained using methanol as the organic solvent-containing solution. The term “SU-ETH” indicates a final liquid extract obtained using ethanol as the organic solvent-containing solution. The indications “S1”, “S2” and “S3” indicate a first final liquid extract, a second final liquid extract, and a third final liquid extract, respectively, using the indicated solvents as the organic solvent-containing solutions or purified water.

The results demonstrate that both aqueous and non-aqueous organic solvent containing solutions may be used to recover biologically active components exhibiting at least anti-MMP activity from shark cartilage. Moreover, residual activity may be extracted by successive re-extraction of the solid particles of shark cartilage. There is no apparent direct correlation between anti-MMP activity and the amount of material isolated, as determined by dry weight analysis and protein recovery.

Impact of Cartilage to Purified Water Ratios on the Production of Liquid Extracts

According to a first embodiment of the process of the invention, the crude liquid extract is prepared with water at a cartilage (C) to purified water (E) ratio of about 1 kg to 1 L, respectively. The process for recovering the components comprised the steps of:

a) homogenizing shark cartilage in an aqueous solution until the cartilage is reduced to solid particles having an average particle size of less than about 500 microns to form a homogenate;

b) equilibrating said homogenate to extract biologically active components into said aqueous solutions to form a first mixture comprising a first mass of solids and a first liquid extract (LE) containing said biologically active components;

c) separating said first liquid extract from said first mass of solids;

d) subjecting said first liquid extract to a separation procedure to form a second liquid extract containing cartilage molecules having molecular weights less than about 500 kDa (LE-0-500);

e) filtering said second liquid extract through a microfiltration membrane having a nominal porosity of 0.22 microns to form a final liquid extract (P-C1-E1 which is substantially equivalent to the 0-500 fraction);

The present process has also been performed using different cartilage to water ratios as follows:

| Fraction ID | Qty of cartilage (Kg) | Qty of purified water (L) |
|---|---|---|
| *P-C3-E1 | 3 | 1 |
| P-C2-E1 | 2 | 1 |
| P-C1-F1 | 1 | 1 |
| P-C1-E2 | 1 | 2 |
| P-C1-E3 | 1 | 3 |

*P indicates the permeate formed during the separation step.

All the first liquid extracts prepared according to the above procedures were analyzed for their dry weight content, protein concentration and their anti-MMP activity. The results are summarized in Table 2.

TABLE 2

| Fractions | Dry weights | Protein | GIA* |
|---|---|---|---|
| tested | (mg/ml) | concentration (μg/ml) | (% of inhibition) |
| P-C3-E1 | 25.2 | 482.5 | 55 |
| P-C2-E1 | 22.1 | 379.4 | 52 |
| P-C1-E1 | 15.0 | 324.3 | 54 |
| P-C1-E2 | 9.9 | 191.5 | 32 |
| P-C1-E3 | 6.3 | 157.8 | 24 |

*GIA was performed on 30 μl aliquots of 20X concentrated samples.

These results indicate that about 20 g of soluble components can be recovered per kilogram of shark cartilage starting material. The maximum recovery of soluble components under the specified conditions were 19.8 (9.9×2) and 18.9 (6.3×3) g of soluble component per kg of shark cartilage (P-C1-E2 and P-C1-E3, respectively).

These results also indicate that the dry weight content, the protein content as well as the components possessing the anti-MMP activity can be efficiently recovered using different cartilage to purified water ratios.

The first solid mass recovered from the P-C1-E1 extraction was re-extracted for 2 more times using the same cartilage to purified water ratio to recover the residual amounts of components contained therein. The process of repeated extraction of the first mass of solids comprises the steps of:

f) treating said first mass of solids recovered from step c) with purified water to form a second mixture which is separated to form a second liquid extract (P-C1-E1-2) and a second mass of solids, wherein said second liquid extract can be treated according to steps d) and e); and, optionally

g) repeating step f) with said second mass of solids to form a third liquid extract (P-C1-E1-3) and a third mass of solids, wherein said third liquid extract can be treated according to steps d) and e).

Table 3 summarizes the amount of water and shark cartilage used in steps a) through g) above.

TABLE 3

| Fraction ID | Qty of cartilage (Kg) | Qty of purified water (L) |
|---|---|---|
| P-C1-E1 | 1 | 1 |
| P-C1-E1-2 | mass of solids after | 1 |
| | recovery of P-C1-E1 | |
| P-C1-E1-3 | mass of solids after | 1 |
| | recovery of P-C1-E1-2 | |

All the liquid extracts resulting from the above procedure were analyzed for dry weight content, protein concentration and anti-MMP activity. The results are summarized in Table 4.

TABLE 4

| | | Protein | |
|---|---|---|---|
| | Dry weights | concentration | GIA* (% of |
| Fractions tested | (mg/ml) | (μg/ml) | inhibition) |
| P-C1-E1 | 15.0 | 324.3 | 54 |
| P-C1-E1-2 | 4.3 | 54.5 | 21 |
| P-C1-E1-3 | 1.3 | 27.0 | 17 |

*GIA was conducted on 30 μl aliquots of 20X concentrated samples.

These results indicate that one or more extractions of shark cartilage according to steps a) through c) above can result in increased recovery of the soluble components of the shark cartilage. Moreover, residual amounts of components possessing anti-MMP activity can still be extracted after a second and third extraction of the same solid particles.

It will be apparent to those of skill in the art that modifications to the extraction parameters such as the temperature, the number of extractions or the extracting solvent, for example, can be made to optimize the amounts of recovered solids, protein and biologically active components.

A Process for Preparing Various Molecular Weight Fractions of Components Derived from Cartilage

The 0-500 Fraction:

The 0-500 fraction is a shark cartilage liquid extract comprising components having molecular weights less than about 500 kDa. Preparative methods for the 0-500 fraction are disclosed in International Publication No. WO 95/32722, WO 96/23512, and WO 97/16197, the relevant disclosures of which are hereby incorporated by reference. These prior art methods comprise the steps of:

a) homogenizing shark cartilage in an aqueous solution in conditions compatible with the preservation of the integrity of biologically active components present in cartilage until the cartilage is reduced to solid particles whose size is less than about 500 μm;

b) extracting said biological active components into said aqueous solution, which results in a mixture of solid particles and of crude liquid extract (LE) having said biologically active components;

c) separating said liquid extract from said solid particles;

d) further separating the crude liquid extract so as to obtain a final liquid extract containing molecules having molecular weights less than about 500 kDa (LE-0-500); and

e) filtering the LE-0-500 on a microfiltration membrane (0.22 micron) and freezing to obtain the final liquid extract (0-500 fraction).

The 0-1 and 1-500 Fractions:

The 0-1 fraction is a shark cartilage liquid extract comprising components having molecular weights less than about 1 kDa. The 1-500 fraction is a shark cartilage liquid extract comprising components having molecular weights between about 1-500 kDa. The 0-1 and 1-500 fractions of shark cartilage extract were prepared with an ultrafiltration system using a membrane having a nominal molecular weight cut-off of about 1 kDa. Using this system, the two cartilage fractions were obtained after one cycle of purification (one cycle of purification being defined by the arrest of the purification step when 50% of the permeate is recovered). The 1-500 fraction comprised the retentate (R) which, when reconstituted using purified water in a final volume equivalent to the original volume of the cartilage extract used for the purification, comprises components having molecular weights of about 1 to 500 kDa at a 1× concentration and components having molecular weights less than about 1 kDa at a 0.5× concentration with regard to the original extract used for the purification. The 0-1 fraction comprised the permeate (P) which is composed only of components having molecular weights less than about 1 kDa at a 1× concentration. Using the ultrafiltration system, the 1-500 fraction was further purified by additional purification cycles as demonstrated in Table 5.

TABLE 5
THEORETICAL CONCENTRATION AFTER SUCCESSIVE ULTRAFILTRATION
ON A PM1

| | | | RETENTATE (R) | | |
|---|---|---|---|---|---|
| CYCLE OF | PERMEATE (P) | | Fraction | | |
| PURIFICATION | Fraction ID | [<1 KDa] | ID | [<1 KDa] | [<1-500 KDa] |
| 1 | P1-0-1 | 1X | R1-1-500 | 0.5X | 1X |
| 2 | P2-0-1 | 0.5X | R2-1-500 | 0.25X | 1X |
| 3 | P3-0-1 | 0.25 | R3-1-500 | 0.13X | 1X |
| 4 | P4-0-1 | 0.13X | R4-1-500 | 0.06X | 1X |
| 5 | P5-0-1 | 0.06X | R5-1-500 | 0.03X | 1X |
| 6 | P6-0-1 | 0.03X | R6-1-500 | 0.02X | 1X |

Multiple batches of the 0-1 and 1-500 fractions were prepared according to the above procedures. In order to minimize the formation of aggregates and to improve the dissolution and the maintenance of a, stable, soluble form, a 1% w/v sucrose aqueous solution was used as a stabilizer for extraction.

The 0-1 and 1-500 fractions were obtained by first preparing a batch of the LE-0-500 fraction according to the prior art methods described above and second adding the following novel steps:

e) optionally preparing the LE-0-500 extract with a solution containing sucrose to a final concentration of about 1% (w/v) to form the LE-0-500 fraction with 1% sucrose;

f) filtering the LE-0-500 or LE-0-500 with 1% sucrose with a membrane having a nominal molecular weight cut-off of about 1 kDa to form liquid extracts comprising cartilage molecules having molecular weights less than about 1 kDa (Pn-0-1 and fraction Pn-0-1 with 1% sucrose, respectively, wherein “n” indicates the purification cycle in Table 5), and to form retentate liquid extracts (Rn-0-1 and fraction Rn-0-1 with 1% sucrose, respectively, wherein “n” indicates the purification cycle in Table 5) comprising cartilage molecules having molecular weights greater than about 1 kDa; and;

g) microfiltering the retentate and permeate liquid extracts through a microfiltration membrane having a porosity of about 0.22 microns.

The above procedure can be performed without including step e) so as to prepare extracts that are free of sucrose. The retentate liquid extracts can be ultrafiltered for one or more, preferably four or more, cycles of purification to form additional filtrate liquid extracts comprising cartilage components having molecular weights less than about 1 kDa (P1-0-1 through P6-0-1) and to form retentate extracts comprising cartilage components having molecular weights between 1 to about 500 kDa (R6-1-500 and R6-1500 with 1% sucrose). The liquid extracts can optionally be frozen for storage.

Accordingly, the procedure just described was used to prepare the following liquid extracts.

1) 0-500 fraction prepared from LE 0-500

2) 0-500 fraction with 1% sucrose prepared from LE-0-500 with 1% sucrose

3) 0-1 fraction prepared from P1-0-1

4) 0-1 fraction with 1% sucrose prepared form P1-0-1 with 1% sucrose

5) 1-500 fraction prepared from R6-1-500

6) 1-500 fraction with 1% sucrose prepared from R6-1-500 with 1% sucrose.

The second mass of solids obtained from the separation of the second mixture which was formed during the treatment of the first mass of solids with water can be repeatedly extracted with water to recover additional amounts of the soluble fraction of shark cartilage.

All liquid extracts prepared according to the above procedure were analyzed for their dry weight and protein content. In addition, the anti-MMP activity as well as the antiangiogenic and the anti-tumor activities of each fraction were also determined. The results are summarized in Table 6.

TABLE 6

| | | PROTEIN | | | |
|---|---|---|---|---|---|
| | DRY | CONCEN- | GIA * | EVT | LLC |
| FRACTIONS | WEIGHTS | TRATION | (% of | (% of | (% of |
| TESTED | (mg/ml) | (μg/ml) | inhibition) | efficacy) | efficacy) |
| Saline | — | — | — | — | 0 |
| 0-500 fraction | 14.8 | 256.1 | 49 | 100 | 32.9 |
| 0-1 fraction | 12.1 | 0.0 | 26 | 80 | 31.0 |
| 1-500 fraction | 0.2 | 163.9 | 21 | 0 | 20.5 |
| 0-500 fraction | 24.7 | 274.5 | 59 | 75 | 42.5 |
| in 1% sucrose | | | | | |
| 0-1 fraction in | 20.3 | 0 | 29 | 100 | 29.2 |
| 1% sucrose | | | | | |
| 1-500 fraction | 11.1 | 212.6 | 14 | 20 | 32.8 |
| in 1% sucrose | | | | | |

* GIA was performed on 30 μl aliquots of 20X concentrated samples.

The analytical results demonstrate that both the 0-1 fraction and the same with 1% sucrose, while containing over 90% of the recovered dry weight content, comprise very low amounts, almost undetectable amounts, of proteins.

However, anti-MMP activity was observed in both the 0-1 fraction as well as the 1-500 fraction suggesting that 1) at least one non-protein component is responsible for this activity, and 2) more than one component may have anti-MMP activity. The active component may or may not be of a protein or peptide nature.

Further, the antiangiogenic activity, as measured according to the EVT, was observed exclusively in the 0-1 fraction. We note that the presence of sucrose was responsible for a slight recovery of antiangiogenic activity in the 1-500 fraction in 1% sucrose.

Treatment of animals, inoculated with M27 tumor cells (LLC), resulted in a significant reduction in the number of macroscopically visible metastatic nodules at the surface of the lung. Both the 0-1 and 0-500 fractions induced a significant reduction in the number of metastatic nodules (about 30%). However, the 1-500 fraction was less active than either the 0-1 or 0-500 fractions suggesting that an active component in the 0-1 fraction is at least partly responsible for the anti-tumor activity. These results also suggest the presence of another anti-tumor component in the 1-500 fraction. Some additional groups of animals have been treated with the same molecular weights fractions containing 1% w/v sucrose. Although the present inventors did not observe any significant difference between groups, there is however a trend for high molecular weights fractions to be more active in the presence of sucrose (above Table). The present inventors did not observe any decrease of animals body weights suggesting the absence of toxicity of the cartilage extract in the LLC model.

Isolation and Characterization of an Anti-MMP Component:

Chromatographic Isolation and Purification

Having found that a plurality of components possessing useful biological activities are present in the 0-500 fraction and more specifically in the 0-1 fraction, the next step was to isolate active components therefrom.

Four different procedures were developed to isolate and purify components containing anti-MMP activity from the 0-500 fraction.

Procedure 1:

Step 1:

The 0-500 fraction obtained by the above detailed procedure was lyophilized and reconstituted (to a 20-fold concentration with regard to the original volume) in purified water. The reconstituted material was sonicated for 15 minutes to optimize solubilization of biologically active components. After a separation procedure, such as centrifugation at 2200 g for 10 min at 4° C., the supernatant was kept for further purification.

Step 2:

Adsorptive chromatography using a solid phase extraction column (SPE-C18 neutral) was performed.

An SPE column packed with 500 mg of C18 sorbent (Supelco No. 5-7012, dimension 3 cc) was conditioned two times in 2 ml of methanol (100%) and three times in 2 ml of purified water. One ml of the 20× reconstituted cartilage extract was loaded onto the column. The sorbent bead was washed with 1.5 ml of purified water, and the components possessing anti-MMP activity were eluted with two 2.5 ml portions of purified water which were combined to form a first eluant.

About 50% of the anti-MMP initial activity was recovered in the first eluant. The remaining 50% was lost during the column loading and washing steps. Neutral conditions therefore appeared to provide weak retention of components possessing the anti-MMP activity. Weak retention of the components, while using this chromatographic medium, is indicative of polar or ionic components.

Step 3:

After repeating the above process a plurality of times with various samples of 20× reconstituted cartilage extract, the respective first eluants were pooled and evaporated on a Speed Vac centrifuge. The solids obtained therefrom were reconstituted in purified water at a 200-fold concentration with regard to the original volume of the 0-500 fraction used. After sonication and centrifugation, the supernatant was kept for the next step of purification.

Step 4:

A low resolution semi-preparative HPLC separation of the biologically active components present in the supernatant was performed in neutral conditions. A Novapack C18HR (7.6×300 mm; Waters) column was used. The mobile phase used was sodium phosphate (0.01 M pH 7)/methanol (92:8). The flow rate and temperature were maintained at 2 ml/minute and 30° C., respectively. The above 200× reconstituted fraction (100 μl) was injected onto the column and 2 ml fractions were collected using isocratic elution conditions and UV detection (205 nm). The running time was 30 minutes. Components possessing anti-MMP activity were found in eluant fractions corresponding to those having a retention time between 11 and 13 minutes.

Step 5:

Step 4 was repeated with various 100 μl aliquots of the 200× reconstituted fraction and the corresponding desired eluant fractions pooled, evaporated, reconstituted in purified water, at a 500× concentration with regard to the original volume of the 0-500 fraction used, and sonicated and centrifuged. The supernatant was kept for the next step of purification.

Step 6:

A higher resolution semi-preparative HPLC in neutral conditions was performed on the supernatant obtained from Step 5. The procedure used for this higher resolution semi-preparative HPLC resembles that of step 4 above except that the phosphate buffer (0.01 M, pH 7)/methanol (97:3) is used as the mobile phase. Components possessing anti-MMP activity were found in eluant fractions corresponding to those having a retention time between 23 and 27 minutes.

Step 7:

After repeating step 6, pooling the corresponding eluant fractions containing active components and evaporating the solvent to form a substantially solid residue, the residue was reconstituted in water, at a 500-2000× concentration with regard to the original volume of the 0-500 fraction used, and sonicated and centrifuged and kept for further molecular weight analysis and determination of its anti-MMP activity. The biologically active component was termed “ [symbol] -986”.

Procedure 2:

It was determined that generally a better retention of [symbol] -986 on the C18 phase chromatography medium was observed at pH 3. Therefore, the SPE procedure (step 2 above) as well as the semi-preparative chromatographic system (steps 4 and 6 above) were modified. The conditions below allow the use of a stronger washing solution in the SPE procedure resulting in a cleaner final extract and in the elimination of one of the semi-preparative purification steps (step 4 of procedure 1).

For example, steps 1 to 3 of procedure 1 were repeated. Step 4 was replaced by the following

Step 4:

The same SPE C-18 column as in step 2 above was used, but the chromatographic medium was conditioned three times with 2 ml of ammonium formate (0.01 M, pH 3). One ml of 200× reconstituted extract, obtained from step 3 (pH adjusted to 3 with formic acid prior loading of the samples), was loaded onto the column. The sorbent bed was washed three times with 2 ml ammonium formate/methanol (90:10, at pH 3). Elution of the [symbol] -986 was performed with 1 ml of methanol (100%). It will be apparent to those of skill in the art that fractions obtained from methanolic elution of the column will contain water. Consequently, the eluting solvent in this step can be another organic solvent, preferably a polar and/or water miscible organic solvent, and the eluting solvent can contain water.

Step 5:

Step 5 of procedure 1 was repeated, except that the concentration of the reconstituted anti-MMP fraction was 4000×.

Step 6:

This step is identical to step 6 of procedure 1, except that the mobile phase was ammonium formate/methanol (75:25 pH 3).

Step 7:

Step 7 was the same as step 7 of procedure 1 except that the same concentration of 4000× was kept as described in the preceding step 6.

Procedure 3:

This procedure is substantially the same as procedure 2, except that in step 6 the pH of the formate buffer was changed from acidic (pH 3) to neutral conditions (about pH 7).

Procedure 4:

In this purification procedure, an acidic mobile phase was used from the beginning.

Step 1:

The pH of the original 0-500 fraction (at a 1× concentration) was adjusted to pH 3 with formic acid and then centrifuged for 10 minutes at 2200 g. The supernatant was used in step 2.

Step 2:

The supernatant was loaded onto an SPE C-18 cartridge (Supelco # 5.-7136: dimension 60 cc packed with 10 g of solid phase support) that had been conditioned under acidic conditions. The column was conditioned with 120 ml methanol (100%) and 120 ml formic acid (0.01 M, pH 3). Five hundred ml of IX acidified cartilage extract was loaded onto the column and eluted with six volumes of 100 ml of formic acid (0.01 M (pH 3)/methanol 90:10). Biologically active components were obtained in eluant fractions 3, 4, and 5.

Step 3:

The eluant fractions 3, 4 and 5 of step 2 were pooled and the solvent evaporated to near dryness. The fractions were then diluted to a concentration of 4000× of original to form an [symbol] -986 containing solution.

Step 4:

The [symbol] -986 was purified on a preparative HPLC column in formic acid buffer pH 3. The column (Prodigy OSD-prep, 10 u, 250×50 mm, from Phenomenex) was conditioned and run at room temperature. The composition of the mobile phase was formic acid (0.01 M, pH 3)/methanol (70:30) and the flow rate was 45 ml/min. Four ml of the SPE C-18 fraction at 4000× concentration were injected onto and eluted from the column in an isocratic mode using UV detection (205 nm). Fractions were collected in one minute intervals for 60 minutes. The anti-MMP activity of the [symbol] -986 was eluted between 33 and 36 minutes.

Step 5:

The fractions exhibiting anti-MMP activity were pooled and evaporated to obtain a 10000× concentrated fraction.

Step 6:

This step is identical to step 6 of procedure 2 except that the mobile phase was formic acid (0.01 M, pH 3)/methanol (75:25). Five hundred μl aliquots of the 10000× concentrated fraction were loaded onto the column. Components containing anti-MMP activity were eluted between 21 and 23 minutes.

Step 7:

Step 7 was the same as the step 7 of procedure 1. The same concentration 4000× was preserved as in the preceding step 6.

Semi-purified Fractions Prepared According to Procedure 1:

The present inventors show for the first time that an HPLC-purified fraction (the fraction resulting from procedure 1 described above) has components possessing an anti-MMP activity. The components thus purified also show anti-tumor activity as demonstrated in the in vivo model LLC described above. The anti-tumor activity was determined by treating animals with 3 different concentrations of the HPLC-purified fraction. A bell-shape dose response curve with a maximum efficacy of about 50% (p<0.005) for the 2.5× concentration dose (the concentration being based in a 100% recovery during the purification steps and with regard to the original volume of cartilage extract) was observed.

Since angiogenesis and matrix metalloprotease activity are closely linked to tumor proliferation and metastasis progression, the HPLC purified fraction representing an anti-MMP component may be responsible for the anti-tumor activity. Therefore, components possessing these activities are potential therapeutic agents in the treatment of cancer (Tolnay, E. et al., J. Cancer Res Clin. Oncol. 123: 652-658, 1997; Skobe, M., et al. Nature Medicine, 3: 1222-1227, 1997).

Semi-purified Fractions Prepared According to Procedure 4:

The fractions in this section were prepared according to procedure 4 above except that steps 2) and 3) were conducted as follows.

Step 2

The supernatant was loaded onto an SPE C-18 cartridge (Supelco # 5.-7012: dimension 3 cc packed with 500 mg of solid phase support) that had been conditioned under acidic conditions. The column was conditioned with 4 ml methanol (100%) and 6 ml formic acid (0.01 M, pH 3). Ten ml of 1× acidified cartilage extract was loaded onto the column washed three times with 1.0 mL volumes of formic acid (0.01 M (pH 3)/methanol 90:10) and the biologically active components eluted therefrom with 1.0 mL of methanol.

Step 3:

The eluant fraction of step 2 containing biologically active components was evaporated to dryness. The fractions were then diluted to a concentration of 40× or 20× of original to form an [symbol] -986 containing solution.

All the liquid extracts resulting from this procedure were analyzed for anti-MMP activity. The results are summarized in Table 7.

TABLE 7

| Fractions tested | GIA (% of inhibition) |
|---|---|
| CTRL-S1* | 57 |
| CTRL-S2* | 16 |
| CTRL-S3* | 4 |
| SU-MET-S1* | 55 |
| SU-MET-S2* | 15 |
| SU-MET-S3* | 0 |
| SU-ETH-S1* | 14 |
| SU-ETH-S2* | 1 |
| SU-ETH-S3* | 0 |
| 0-500 fraction** | 64 |
| 0-1 fraction** | 56 |
| 1-500 fraction** | 16 |
| 0-500 fraction in 1% sucrose** | 74 |
| 0-1 fraction in 1% sucrose** | 40 |
| 1-500 fraction in 1% sucrose** | 16 |
| P-C3-E1* | 57 |
| P-C2-E1* | 60 |
| P-C1-E1* | 46 |
| P-C1-E2* | 39 |
| P-C3-E3* | 17 |
| P-C1-E1-2* | 16 |
| P-C1-E1-3* | 4 |

*GIA was performed on 80 μl aliquots of 20X concentrated samples.
**GIA was performed on 80 μl aliquots of 40X concentrated samples.

Thus, the process of the present invention provides for the preparation of specific shark cartilage fractions possessing anti-MMP activity. Further, both aqueous and organic solvent-containing solutions can be used to prepare cartilage extracts possessing at least an anti-MMP activity. Although both of the 0-500 and 1-500 fractions have anti-MMP activity, anti-MMP components purified by the present procedure are mainly contained within the 0-1 kDa portion. Similar results have been observed in the equivalent fractions containing 1% w/v sucrose. Finally, the anti-MMP activity can be efficiently recovered using different cartilage to purified water ratios.

Recovery of Activities in Different Solvents:

The results obtained with organic solvent-containing solutions, namely ethanol and methanol, encouraged the present inventors to test many other solvents and verify the inhibitory activities in enzymatic, proliferation and angiogenic assays of extracts recovered from these different solvents.

The activities of the cartilage extract were tested in the following assays:

Gelatinase Inhibition Assay (MMP-2):

In order to characterize the ability of the liquid cartilage extract to inhibit the activity of metalloproteases, a gelatinase inhibition assay (GIA) has been performed using a commercial kit (Boehringer Mannheim). Briefly, a biotin-labeled gelatin substrate is incubated with gelatinase A (MMP-2) in the absence or the presence of the liquid cartilage extract or its derivatives. Subsequently, the reaction mix was loaded onto a streptavidin-coated microtiter plate. The biotin-labeled gelatin binds to the streptavidin-coated microtiter via its free biotin residues of the biotin-labeled gelatin. If the substrate, gelatin, is not spliced by gelatinase, a streptavidin-peroxidase (POD) conjugate binds to the remaining free biotin residues of the gelatinase-biotin-complex. POD then converts the added ABTS substrate to a green end product, which can be measured at 405 mm. However, if the biotin-labeled gelatin is spliced by gelatinase before, only small fragments occur with one biotin residue each. After the attachment to the microtiter plate, these fragments do not have the capacity to bind the streptavidin-POD conjugate and non colored reaction occurs.

Elastase Inhibition Assay (PPE):

In order to characterize the ability of the liquid cartilage extract to inhibit the activity of metalloproteases, an elastase inhibition assay has been performed using a slightly modified commercial kit (Molecular Probes). Briefly, a soluble elastin substrate (from bovine neck ligament) (6.25 μg/ml) conjugated with a fluorochrome is incubated with porcine pancreatic elastase (PPE; 0.0125 U/ml) in the absence or the presence of a shark cartilage extract or its derivatives. Upon digestion by elastase, the fluorescence is revealed and emission is measured with a fluorescence microplate reader (505 to 515 nm). In the presence of an inhibitor of elastase such as any one present in the liquid cartilage extract, elastin digestion is prevented and fluorescence emission inhibited.

In vitro Endothelial Cell Proliferation Assay (HUVEC):

In order to characterize the ability of the cartilage extract to inhibit the proliferation of endothelial cell in vitro, an assay based on the quantification of cell proliferation was performed. Cryopreserved human umbilical vein endothelial cells (HUVECs) used were obtained from a commercial source and were tested for mycoplasma and some viral contamination. HUVECs were thawed and cultured according to the manufacturer's directives. In preparation for the assay, HUVECs were seeded at 4,000 cells/well in 96-well sterile culture dishes. After allowing cell attachment within for 6-8 hours, fresh medium containing different concentrations of the shark cartilage extract, its derivatives, negative and positive controls were added to the cell cultures. Cells were then incubated for a period of 3 days at 37° C. in the presence of the appropriate test article as described above. After that 3-day period, cell number was evaluated by DNA staining using Hoescht-33257 as fluorescent dye. Decreased cell number was an indication of an inhibitory effect on HUVECs proliferation.

Differences in the Compositions of the Extracts Obtained from Different Solvents:

(HPSEC): High Pressure Size Exclusion Chromatography

Vector Angle and Ratio Length:

In order to compare complex spectra generated by each extracts, we used the vector angle. This approach is universally applicable to data sets consisting of paired data values (Brown and Donahue (1988) Applied Spectroscopy. 42(2): 347). In the case of chromatograms, the detector signal (in this case UV; 205 nm) is measured at periodic intervals after injection and the data obtained from such chromatograms form the data base for the comparison. The angle between two given vectors is a measure of the difference between the two chromatographic patterns, regardless of overall spectral intensity. A perfect match between the spectra yields an angle of 0. With vector angle comparison, one may determine whether two different chromatograms have the same “pattern” of peak but not whether they differ in intensity. To evaluate intensity difference, an additional statistical tool has been developed to compare the length of the vectors. This tool utilizes the ratio of spectra length. The perfect match for length ratios between different spectra yields a value of 1.0.

Determination of Protein Concentration Using Bradford Assay:

Analysis of the test article for the determination of the protein content is performed with a standard assay for microtiter plate. Briefly, proteins of samples and of solutions of IgGB standard (bovine gamma globulin) of concentrations ranging to 200 μg/ml to 800 μg/ml are solubilized with 0.03N final of NaOH. 20 μl of each sample and standard are added to triplicate wells of a microtiter plate, 200 μl of dye reagent (Coomassie Brilliant Blue G-250 diluted 1/5) is added to each well. The absorption at 595 nm is determined after 5 minutes of incubation using a plate reader.

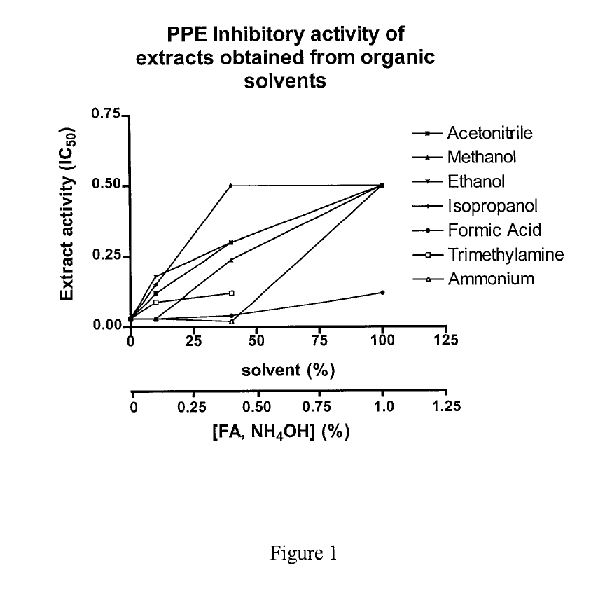

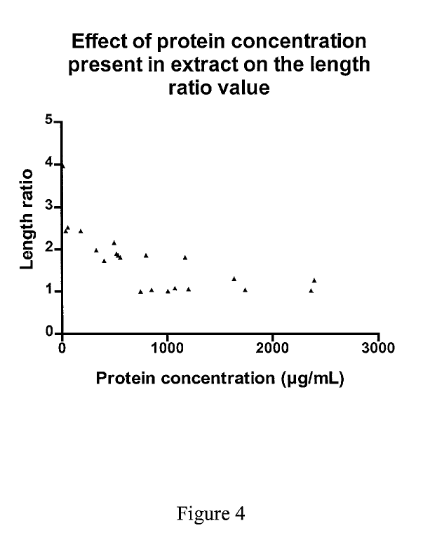

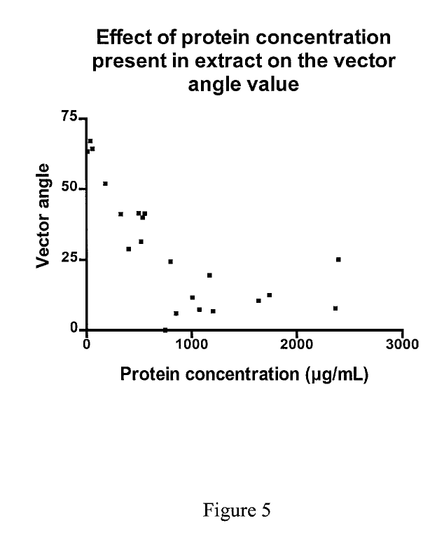

The results of the investigation of the recovery profile and the activities recuperated in the extracts obtained with different categories of solvents are shown in Tables 8 to 11. The behavior of the recovery of the biological compounds in different solvents is shown in FIGS. 1 to 3. The comparative compositions in different solvents are shown in FIGS. 4 and 5.

TABLE 8
Aprotic solvents

| | | | | | | HPSEC | |
|---|---|---|---|---|---|---|---|
| | | MMP-2 | PPE | HUVEC | | (vector angle) | |
| | | (IC₅₀) | (IC₅₀) | (IC₅₀) | Protein | An- | Length |
| Solvents | | (μg/ml) | (μg/ml) | (μg/ml) | (μg/ml) | gle | ratio |
| ACN | 100% | 0.15 | >0.5 | 0.28 | <12.5 | 63.3 | 3.97 |
| | 40% | 0.02 | 0.3 | 0.26 | 799 | 24.4 | 1.86 |
| | 10% | 0.02 | 0.12 | 0.30 | 1203 | 6.8 | 1.06 |
| DMSO | 100% | 0.05 | N.D. | >1 | 379 | | |
| H₂O | 100% | 0.02 | 0.03 | 0.48 | 745 | 0 | 1.0 |

TABLE 9
Protic solvents

| | | | | | | HPSEC | |
|---|---|---|---|---|---|---|---|
| | | MMP-2 | PPE | HUVEC | | (vector angle) | |
| | | (IC₅₀) | (IC₅₀) | (IC₅₀) | Protein | An- | Length |
| Solvents | | (μg/ml) | (μg/ml) | (μg/ml) | (μg/ml) | gle | ratio |
| MetOH | 100% | 0.13 | 0.5 | 0.18 | 37 | 67.07 | 2.43 |
| | 40% | 0.02 | 0.24 | 0.20 | 534 | 39.97 | 1.86 |
| | 10% | 0.02 | 0.03 | 0.16 | 851 | 6.01 | 1.04 |
| EtOH | 100% | 0.08 | 0.05 | 0.17 | 57 | 64.29 | 2.52 |
| | 40% | 0.03 | 0.03 | 0.20 | 554 | 41.41 | 1.81 |
| | 10% | 0.02 | 0.18 | 0.19 | 1006 | 11.74 | 1.01 |
| IsopOH | 100% | 0.06 | >0.5 | 0.22 | 179 | 52.02 | 2.43 |
| | 40% | 0.02 | 0.5 | 0.27 | 326 | 41.22 | 1.98 |
| | 10% | 0.01 | 0.15 | 0.28 | 2396 | 25.11 | 1.26 |
| H₂O | 100% | 0.02 | 0.03 | 0.48 | 745 | 0 | 1.0 |

TABLE 10
Acid solvents or solutions

| | | | | | | HPSEC | |
|---|---|---|---|---|---|---|---|
| | | MMP-2 | PPE | HUVEC | | (vector angle) | |
| | | (IC₅₀) | (IC₅₀) | (IC₅₀) | Protein | An- | Length |
| Solvents | | (μg/ml) | (μg/ml) | (μg/ml) | (μg/ml) | gle | ratio |
| Formic | 1% | 0.03 | 0.12 | 0.13 | 496 | 41.56 | 2.16 |
| Acid | 0.4% | 0.02 | 0.04 | 0.16 | 400 | 28.93 | 1.73 |
| | 0.1% | 0.02 | 0.03 | 0.30 | 518 | 31.41 | 1.90 |
| TFA | 1% | | N.D. | 0.43 | 336 | | |
| H₂O | 100% | 0.02 | 0.03 | 0.48 | 745 | 0 | 1.0 |

TABLE 11
Basic solvents or solutions

| | | | | | | HPSEC | |
|---|---|---|---|---|---|---|---|
| | | MMP-2 | PPE | HUVEC | | (vector angle) | |
| | | (IC₅₀) | (IC₅₀) | (IC₅₀) | Protein | An- | Length |
| Solvents | | (μg/ml) | (μg/ml) | (μg/ml) | (μg/ml) | gle | ratio |
| Tri- | 40% | 0.01 | 0.12 | 0.02 | 1636 | 10.52 | 1.30 |
| methyl | 10% | 0.02 | 0.09 | 0.09 | 2366 | 7.86 | 1.02 |
| amine | | | | | | | |
| (TMA) | | | | | | | |
| NH₄OH | 1% | 0.02 | >0.5 | 0.70 | 1073 | 7.40 | 1.08 |
| | 0.4% | 0.02 | 0.02 | 0.19 | 1740 | 12.64 | 1.40 |
| | 0.1% | 0.03 | 0.03 | 0.43 | 1171 | 19.51 | 1.81 |
| H₂O | 100% | 0.02 | 0.03 | 0.48 | 745 | 0 | 1.0 |

Conclusions:

Matrix metalloproteinases (MMPs) are a family of endopeptidases that collectively cleave most if not all of the constituents of the extracellular matrix. They play a significant role in regulating angiogenesis, the process of new blood vessel formation. They also play an important role in cancer metastasis by favoring local proteolysis of the basement membrane that leads to the invasion of cancer cells into the stroma, followed by an invasion to the capillary cell wall to enter blood circulation. After entering into the blood circulation, these tumor cells migrate to and invade distant target organs. Here, we evaluate the inhibitory activity of various extracts on two different proteolytic enzymes: the MMP-2 and PPE. The MMP-2 is matrix metalloproteinase-2 which has a gelatinolytic activity. The PPE is the porcine pancreatic elastase. Since it is a proteolytic enzyme having an elastinolytic activity, any effect of the extract(s) on PPE should be indicative of an effect on MMPs, enzymes with elastinolytic activity comprising MMP-9.

All cartilage extracts obtained from different organic solvents showed significant inhibitory activities. The concentrations of extract able to inhibit 50% of the PPE activity (IC₅₀) range from 0.02 to 0.5 μg/ml (μg of dry weight/mL) as shown in FIG. 1. Cartilage extract made with water shows an IC₅₀ of 0.02 μg/mL. Similar activity was monitored in extracts obtained with either 10% methanol, 0.1% formic acid or 0.1% ammonium. The anti-PPE activity found in these extracts was less potent as the concentration of organic solvent used for the extraction increased. These results could reflect a decrease in protein concentration monitored in these extracts. However, in the case of formic acid and ammonium, the decrease of anti-PPE potency observed was not linked to a difference in protein concentration, since they are almost identical in each condition of preparation. It is interesting to mention that the activity of MMP-2 is not perturbed by the presence of high concentrations of formic acid or ammonium, the IC₅₀ being 0.03 μg/mL, which value represents about the same potency obtained with an extracts made with pure water. This indicated that the anti-PPE is sensitive to pH variation. Moreover, these results show new methods for the preparation of cartilage extracts having significant anti-MMP-2 with lower anti-PPE activity.

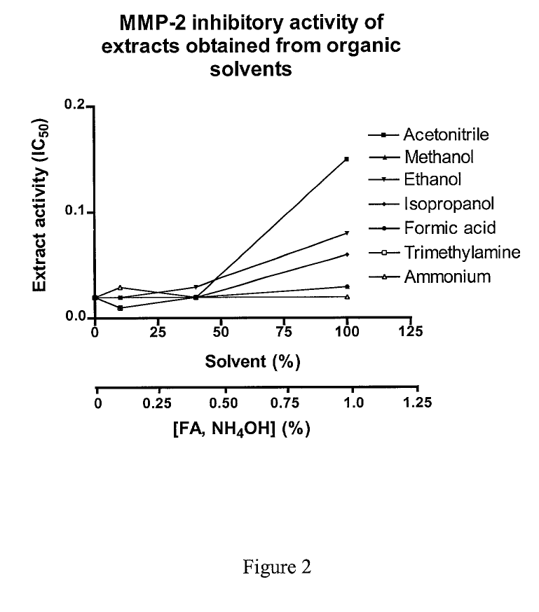

As illustrated in FIG. 2, all cartilage extracts show anti MMP-2 activities, their IC₅₀ ranging from 0.01 to 0.15 μg/mL. An IC₅₀ of 0.02 μg/mL was observed with the reference extracts obtained with pure water. The potency of these extracts seems dependent on protein concentration as observed with PPE inhibition.

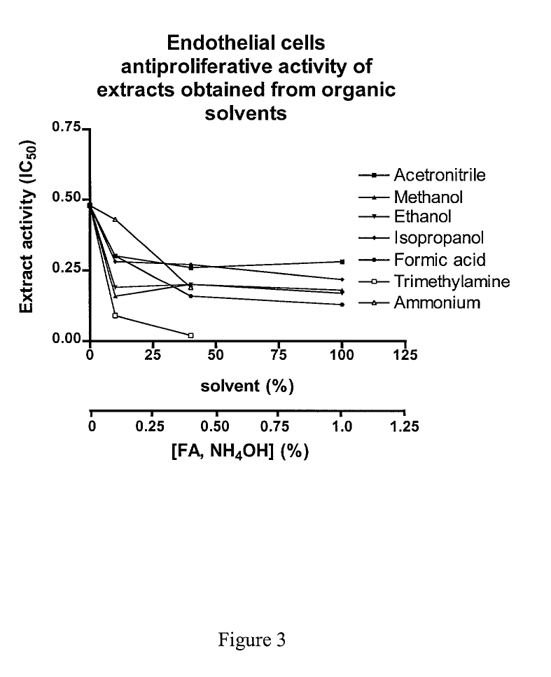

Angiogenesis is a complex process which involved not only MMP but also both endothelial cell proliferation and differentiation. The effect of various cartilage extracts on human umbilical vein endothelial cells (HUVEC) proliferation was established to evaluate their respective antiangiogenic activity. As illustrated in FIG. 3, the antiproliferative activity of these extracts (IC₅₀) varies from 0.02 to 0.5 μg/mL. The activity obtained with the reference cartilage extract made with water was 0.48 μg/mL and the presence of organic solvent during the extraction step generated more active extracts. The most potent extracts were made with trimethylamine (IC₅₀ of 0.09 and 0.02 μg/mL observed for an extract made with 10% and 40% TMA, respectively). These unexpected results indicate an advantage of using this solvent over water to preferentially concentrate bioactive components having HUVEC anti proliferative activity and anti angiogenic activity as well. It is also interesting to mention that the anti proliferative activity of these extracts is not dependent on protein concentration, thus suggesting that non proteinacous component(s) could be responsible of this anti HUVEC activity.

These examples suggested that each of these extracts are different: they have various concentrations of proteins (from about 0 to 1203 mg/mL), and show various patterns of activity in MMP-2, PPE and HUVEC. This is supported by a high pressure size exclusion chromatography (HPSEC) analysis of these extracts using the extract generated with water as reference. This method indicates that the vector angle varies from 0 to about 60 and the length ratio varies from 1 to 4. As expected, the differences increase with a variation in protein concentrations (FIGS. 4 and 5). Moreover, extracts with properties similar to those of the reference extract using pure water as solvent show significant difference. For example, extracts made with 10% methanol show about the same biological activity of pure water, but they show quite important difference in their chromatographic profile (angle=6.01, length ratio 1.04). Conversely, extract with ammonium shows almost the same chromatographic profile as methanol (angle=7.40, length−1.08), but show quite different activities, the anti PPE activity being considerably reduced.

Conclusion:

Extraction with all the tested solvents generated active extracts. However, the inhibition of PPE and MMP-2 is reduced compared to the one of an extract made with water. Conversely, HUVEC activity is higher when organic solvents are used for extraction. TMA extraction generated the overall highest active extract. Therefore, it can be concluded that a great diversity of solvents can be used to extract biologically active components from cartilage. Among those specifically tested, water 100% and TMA 40% were the most performing. Further, using acidic or basic solvents generated extracts with reduced anti-PPE activity. In any way, various degrees of enrichment in some components are obtained in different solvents. The extracts of this invention are capable of influencing biological processes involved in tumor development. Since MMPs and endothelial cell proliferation are key events in angiogenesis, the present extracts should have an activity against neovascularization, and particularly against tumor vascularization and metastasis.

The present process applies to any source of cartilage (from birds, marsupials, batracians, reptiles, mammalian and fishes), although shark cartilage has been preferred.

Molecular Weight Determination of the Anti-MMP Component by LC/MS

Five multi-dimensional chromatographic systems were developed to facilitate the determination of molecular weight of shark cartilage fractions by liquid chromatography/mass spectrometry (LC/MS). Each of five systems is presented below in Tables 12-16.

The experiments involve MS Scanning of the split (7:1) chromatographic column eluant as well as fraction collection from the LC to be used for post-ran anti-MMP activity determinations. This association between MS and anti-MMP biological activity specifically identifies the elution fraction as well as the retention time of the compound of interest for each of the chromatographic system used.

For MS negative ions detection, a solution of ammonium hydroxide (0.75% v/v at 0.15 m/min.) was added to the column eluant prior to introduction into the MS ion source. The resulting pH of the mixture was between 8 to 10 which improve MS negative ions formation and detection.

TABLE 12
CHROMATOGRAPHIC SYSTEM 1: Isocratic C18 neutral condition
(ammonium formate)

| | |
|---|---|
| Column | C18 ODS-2, 5u, 4.6 × 250 mm, Phenomenex |
| Column temperature | 30° C. |
| Flow rate | 0.7 ml/min. |
| Injection volume | 100 μl of purified fraction |
| Eluant | Ammonium formate (0.01 M, pH 7)/methanol (96:4) |
| Elution mode | Isocratic |
| Detection | UV: 205 nm, 254 nm, MS |
| Run time | 25 min. |
| Fraction collection | each min. or 30 sec. with different delay time. |

Anti-MMP activity of the collected fractions was evaluated.

TABLE 13
CHROMATOGRAPHIC SYSTEM 2: Gradient C18 acid condition
(ammonium formate)

| Column | C18 ODS-2, 5u, 4.6 × 250 mm, Phenomenex | | |
|---|---|---|---|
| Column temperature | 30° C. | | |
| Flow rate | 0.7 ml/min. | | |
| Injection volume | 100 μl of purified fraction | | |
| Eluant A | Ammonium formate (0.01M, pH 3)/methanol (96:4) | | |
| Eluant B | Methanol | | |
| Gradient | Time | Eluant A | Eluant B |
| | 0 | 100 | 0 |
| | 2 | 100 | 0 |
| | 22 | 20 | 80 |
| | 25 | 20 | 80 |

| | |
|---|---|
| Detection | UV: 205 nm, 254 nm, MS |
| Run time | 25 min. |
| Fraction collection | each min. or 30 sec. with different delay time. |

Anti-MMP activity of the collected fractions was evaluated.

TABLE 14
CHROMATOGRAPHIC SYSTEM 3: Isocratic C18 acid condition
(ammonium formate)

| | |
|---|---|
| Column | C18 ODS-2, 5u, 4.6 × 250 mm, Phenomenex |
| Column temperature | 30° C. |
| Flow rate | 0.7 ml/min. |
| Injection volume | 100 μl of purified fraction |
| Eluant | Ammonium formate (0.01M, pH 3)/methanol |
| | (75:25) |
| Elution mode | Isocratic |
| Detection | UV: 205 nm, 254 nm, MS |
| Run time | 25 min. |
| Fraction collection | each min. or 30 sec. with different delay time. |

Anti-MMP activity of the collected fractions was evaluated.

TABLE 15
CHROMATOGRAPHIC SYSTEM 4: Gradient NH₂ acid condition
(ammonium formate)

| Column | NH₂, 5u, 3.6 × 250 mm, Phenomenex | | |
|---|---|---|---|
| Column temperature | 30° C. | | |
| Flow rate | 0.7 ml/min. | | |
| Injection volume | 100 μl of purified fraction | | |
| Eluant A | Ammonium formate (0.01 M, pH 3)/methanol (96:4) | | |
| Eluant B | Methanol | | |
| Gradient | Time | Eluant A | Eluant B |
| | 0 | 100 | 0 |
| | 2 | 100 | 0 |
| | 22 | 20 | 80 |
| | 25 | 20 | 80 |

| | |
|---|---|
| Detection | UV: 205 nm, 254 nm, MS |
| Run time | 25 min. |
| Fraction collection | each min. or 30 sec. with different delay time. |

Anti-MMP activity of the collected fractions was evaluated.

TABLE 16
CHROMATOGRAPHIC SYSTEM 5: Isocratic C18 acid condition
(ammonium formate)

| | |
|---|---|
| Column | C18 ODS-2, 5u, 4.6 × 250 mm, Phenomenex |
| Column temperature | 30° C. |
| Flow rate | 0.7 ml/min. |
| Injection volume | 100 μl of purified fraction |
| Eluant | Ammonium formate (0.01M, pH 3)/methanol |
| | (75:25) |
| Elution mode | Isocratic |
| Detection | UV: 205 nm, 254 nm, MS |
| Run time | 25 min. |
| Fraction collection | each min. or 30 sec. with different delay time. |

Anti-MMP activity of the collected fractions was evaluated.

The multidimensional chromatographic experiments were conducted by injecting 100 μl of 500 to 1000× of the purified phosphate final fraction (obtained from step 7 of purification procedure 1). At this concentration, no strong and clear signal of the [symbol] -986 was detected in the MS scan mode (total ions). Peaks of interest were detected by post run monitoring all the individual ion signal (100-1000 amu) in the region of interest (active fractions).

Injection of purified fractions with concentrations of up to 2000× showed a small peak in the total ion chromatogram as well as in the base peak chromatogram corresponding to the [symbol] -986.

In positive ion detection mode (Table 17) only ions 245 M+1 and 227 were clearly detected in the region of interest ( [symbol] -986). As per the design and the operation in the LCQ MS, the observation of ions corresponding to the loss of a molecule of water as well as the molecular ion (M+1) is usual and frequent for an analyte containing an alcohol functional group. The co-elution profile of the ions 245 M+1 and 227 as well as the 18 amu difference corresponding to the loss of a molecule of water (H₂O), strongly suggest the presence of a single component of interest with a molecular weight of 244, 245 being equivalent to the M+1 species in positive ion mode.

The post-run analysis of those chromatograms indicated the presence of the ion 245 (M+1) in each of the fractions collected from the different chromatographic systems which contain components possessing anti-MMP activity.

The [symbol] -986 was detected in fractions collected between 13.5 to 15.0 minutes corresponding to a 14.14 minutes retention time for elution of the m/e 245 M+1 peak, on the HPLC C18 system (ammonium formate neutral pH 7 isocratic).

The [symbol] -986 was detected in fractions collected between 16.5 to 17.0 minutes corresponding to a 16.62 minutes retention time for elution of the m/e 245 M+1 peak, on the HPLC C18 system (ammonium formate acid pH 3 gradient).

The [symbol] -986 was detected in fractions collected between 16 to 18 minutes corresponding to a 16.79 minutes retention time for elution of the m/e 245 M+1 peak, on the HPLC C18 system (ammonium formate neutral pH 3 isocratic).

The [symbol] -986 was detected in fractions collected between 14 to 16 minutes corresponding to a 14.28 minutes retention time for elution of the m/e 245 peak, on the HPLC NH2 system (ammonium formate acid pH 3 gradient).

In negative mode (Table 18) only, ions 243 and 289 were detected in the region of interest ( [symbol] -986) in all the chromatographic system evaluated. Again perfect co-elution of those two ions suggest the formation of a formate adduct on the ion 243. This phenomenon is observed frequently in negative ion when ammonium formate is used as buffer in the mobile phase. This was proven by replacing the ammonium formate buffer with an ammonium acetate buffer at the same pH. The ammonium acetate mobile phase was post column alkalinized with ammonium hydroxide solution prior to MS detection. Both systems showed a clear signal for the ion 243 but ion 289 was only detected in the formate system and a new ion (303) corresponding to an acetate adduct was detected in the second chromatographic system. Accordingly, it is believed that the [symbol] -986 component has a molecular weight of about 244 amu (243 equivalent to the M-1 species in the negative ion mode).

TABLE 17
Positive ion detection

| | CHROMATOGRAPHIC CONDITION | | | | | | |
|---|---|---|---|---|---|---|---|
| | ISOCRATIC | ISOCRATIC | ISOCRATIC | GRADIENT | ISOCRATIC | GRADIENT | ISOCRATIC |
| | C 18 | C 18 | C 18 | C 18 | C 18 | NH2 | C 18 |
| | NEUTRAL | NEUTRAL | NEUTRAL | ACID | ACID | ACID | ACID |
| | CONDITION | CONDITION | CONDITION | CONDITION | CONDITION | CONDITION | CONDITION |
| | (AM. | (AM. | (AM. | (AM. | (AM. | (AM. | (AM. |
| DESCRIPTION | FORMATE) | FORMATE) | FORMATE) | FORMATE) | FORMATE) | FORMATE) | FORMATE) |
| Fraction collection | Collection 2 | Collection 3 | Collection 5 | Collection 7 | Collection 9 | F.P.4 | Collection 10 |
| | F1:15 to 1.5 min. | F1:15 to | F1:12 to | F1:12 to | F1:6 to 7 min. | F1:7 to 8 min. | F1:6 to 6.5 min. |
| | | 15.5 min. | 12.5 min. | 12.5 min. | | | |
| | F20:24.5 to | F20:24.5 to | F20:21.5 to | F20:21.5 to | F20:23 to | F15:21 to | F20:25.5 to |
| | 25 min. | 25 min. | 22 min. | 22 min. | 24 min. | 22 min | 26 min. |
| GIA activity | ND | N.E. | 13.5 to 14 | 16.5 to 17 | 16 to 17 | 14 to 15 | 16 to 16.5 |
| | | | min: 49 | min: 36 | min: 32 | min: 68 | min: 12 |
| | | | 14 to 14.5 | | 17 to 18 | 15 to 16 | 16.6 to 17 |
| | | | min: 24 | | min: 13 | min: 8 | min: 29 |
| | | | 14.5 to 15 min: 8 | | | | 17 to 17.5 min: 8 |
| Expected R.T. | 13 to 15 min. | 13 to 15 min. | 14 min. | 16.5 min. | 16.8 min. | 14.5 min. | 16.8 min. |
| m/e Detect | 191 | 191 | 191 | — | — | — | — |
| | 227 | 227 | 227 | 227 | 227 | 227 | 227 |
| | 229 | 229 | 229 | 229 | 229 | — | 229 |
| | 245 | 245 | 245 | 245 | 245 | 245 | 245 |
| | 334 | 334 | 334 | — | — | — | — |
| | 346 | 346 | — | — | — | — | — |
| | 684 | 684 | 684 | 684 | — | | — |
| | 706 | 706 | 706 | 706 | — | | — |

TABLE 18
Negative ion detection

| | CHROMATOGRAPHIC CONDITION | | | | | | |
|---|---|---|---|---|---|---|---|
| | ISOCRATIC | ISOCRATIC | ISOCRATIC | ISOCRATIC | | ISOCRATIC | ISOCRATIC |
| | C 18 | C 18 | C 18 | C 18 | GRADIENT NH2 | C 18 | C 18 |
| | NEUTRAL | NEUTRAL | NEUTRAL | ACID | ACID | ACID | ACID |
| DESCRIP- | CONDITION | CONDITION | CONDITION | CONDITION | CONDITION | CONDITION | CONDITION |
| TION | (AM.FORMATE) | (AM.FORMATE) | (AM.FORMATE) | (AM.FORMATE) | (AM.FORMATE) | (AM.FORMATE) | (AM.FORMATE) |
| Fraction | Collection 4 | Collection 6 | Collection 11 | Collection 12 | Collection 13 | F.P.2 | — |
| collection | F1:15 to | F1:12 to | F1:6 to 7 min. | F1:6 to 6.5 min. | F1:6 to 7 min. | F1:7 to 8 min. | |
| | 15.5 min. | 12.5 min. | F18:23 to 24 min. | F34:22.5 to | F22:27 to 28 min. | F17:23 to 24 min. | |
| | F20:24.5 to | F20:21.5 to | | 23 min. | | | |
| | 25 min. | 22 min. | | | | | |
| GIA activity | ND | 14 to 14.5 min:17 | 16 to 17 min:27 | 16 to 16.5 min:7 | 13 to 14 min:14 | 18 to 19 min:69 | N/A |
| | | 14.5 to 15 min:10 | 17 to 19 min:39 | 16.6 to 17 min:17 | 14 to 15 min:1 | | |
| | | 15 to 15.5 min:10 | | 17 to 17.5 min:18 | | | |
| Expected R.T. | 13 to 15 min. | 14 min. | 17 min. | 17 min. | 14 min. | | |
| M/e Detect | 145 | 145 | — | — | — | — | — |
| | 189 | 189 | — | — | 227 | — | — |
| | 243 | 243 | 243 | 243 | 243 | 243 | 243 |
| | 289 | 289 | 289 | 289 | 289 | 289 | — |
| | 682 | 682 | — | — | — | — | — |
| | 683 | 683 | — | — | — | — | — |

Empirical Formula and Partial Structure Elucidation of [symbol] -986

LC-MS Empirical Formula Determination:

Mass spectrometry was used to obtain information regarding the structure of [symbol] -986. Table 19 summarizes the conditions used in the LC-MS analysis of [symbol] -986.

TABLE 19
Chromatographic conditions used for LC-MS partial empirical formula
determination:

| | |
|---|---|
| Column | C18 ODS-2, 5u, 4.6 × 250 mm, Phenomenex |
| Column temperature | 30° C. |
| Flow rate | 0.7 ml/min. |
| Injection volume | 100 μl of purified fraction |
| Eluant | Ammonium formate (0.01M, pH 3)/methanol |
| | (75:25) |
| Elution mode | Isocratic |
| Detection | UV: 205 nm, 254 nm, MS |
| Run time | 25 min. |
| Fraction collection | each min. or 30 sec. with different delay time. |

The determination of the isotopic ratio of 247, 246, 245 was conducted in zoom scan mode to increase the precision on the reading of the weak signal of those ions. The isotopic ratios obtained for the ion 2461245 (A+1 type) and 247/245 (A+2 type) are presented in a table format below.

Ratio of 5.9% of the m/e 247/245 peak heights (A+2 isotopic ratio) strongly suggest the presence of a sulfur and few oxygen atoms on the molecule.

Isotopic ratio of 11.8% for the A+1 elements (m/e 246/245 peak height) can account for up to 10 carbon or a mixture of carbon, nitrogen and sulfur (1) on the molecule.

With a molecular weights of 244 amu only an even number of nitrogen (0, 2, 4) can be present on this molecule.

LC/MSn Structural Elucidation:

A partial elucidation of the structure of the [symbol] -986 was done by conducting tandem mass spectrometry experiments.

TABLE 20
Chromatographic condition used for MSn experiment are described below:

| | |
|---|---|
| Column | C18 ODS-2, 5u, 4.6 × 250 mm, Phenomenex |
| Column | 30° C. |
| temperature | |
| Flow rate | 0.7 ml/min. |
| Injection volume | 100 μl of purified fraction |
| Eluant | Ammonium formate (0.01 M, pH 3)/methanol (75:25) |
| Elution mode | Isocratic |
| Detection | UV: 205 nm, 254 nm, MS |
| Run time | 25 min. |
| Fraction | each min. or 30 sec. with different delay time. |
| collection | |

Tandem mass spectrometry (MS/MS) experiments which were conducted on positive ions for the molecular ion 245 m/e (M+1) showed losses of 18 amu (m/e 227.1) and 36 amu (m/e 209) (minor). Those losses correspond to the loss of one and two molecules of water (—H₂O and —2 H₂O, respectively), indicating the presence of an alcohol and/or diol moiety in [symbol] -986. The actual MS/MS spectrum is presented in FIG. 5.

An MS/MS experiment conducted on the m/e 227 ion resulted in a complex spectrum with many characteristic fragments of the [symbol] -986 chemical structure. Fragments appearing in this spectrum could result from either one or both fragmentation of the m/e 227 ion or fragmentation of other intense ions appearing in this spectrum (i.e. m/e 166 is from fragmentation of the 209 ion). Consequently, further MS/MS experiments were conducted on selected fragments of the m/e 227 ion. The MS/MS spectrum obtained is depicted in FIG. 6.

An MS/MS experiment on the 209 m/e ion (M+1-2H₂O) results from a loss of 60 amu, to give m/e 149 which is characteristic of a loss of carboxylic acid (—CH₃COOH) moiety.

The ion 149 m/e (M+1-2 H₂O—CH₃COOH) was then reanalyzed by MS/MS and the following fragments were obtained: m/e 105, 115, 116 and 134. Loss of 15 from 149 to 134 most likely corresponds to the loss of CH₃. Loss of 33 and 34 are characteristic of the loss of SH and H₂S therefore strongly suggesting the presence of a sulfur-containing group (thiol or thioether) in [symbol] -986. Loss of 44 from m/e 149 to 105 can be due to losses of several different groups.

Chemical Derivatization Structural Elucidation:

The [symbol] -986 was subject to conditions commonly used for the esterification of carboxylic acids as detailed below.

Methylation (HCl/Methanol)

For methylation of purified fractions, the present inventors evaporated 15 μl of a purified fraction (4000×) of [symbol] -986 and added 100 μl of a mixture HCl (12 N):MeOH/(1:99) in a closed vial. The mixture was incubated 60-90 min. at 45° C., then evaporated to dryness and dissolved in 100 μl of water. This solution was injected according to chromatographic conditions used for LC/MS structure elucidation.

Methylation (BF₃/methanol)

For methylation of purified fractions, the present inventors evaporated 15 μl of a purified fraction (4000×) of [symbol] -986 and added 100 μl of BF₃/methanol solution in a closed vial. The mixture was incubated 60-90 min. at 45° C., then evaporated to dryness and dissolved with 100 μl of water. This solution was injected according to chromatographic conditions used for LC/MS structure elucidation.

Dilution of Purified Fractions (4000×)

To verify the recovery of derivatization, the present inventors diluted 15 μl of a purified fraction (4000×) of [symbol] -986 with 85 μl of water. The diluted solution was analyzed according to the chromatographic conditions used for LC/MS elucidation.

Results

Derivatization of the [symbol] -986 component with BF₃/methanol or H+/methanol at 45° C. for one hour resulted in the disappearance of its chromatographic signal, as determined by signal strength at the expected retention time for the of [symbol] -986, by more than 95%. These two reactions are well known for the transformation of carboxylic acid to their corresponding methyl esters. Methylation causes an increase in the molecular weight of the [symbol] -986 as well as an increase of its retention time on the chromatographic system. The concentration of the [symbol] -986 derivatives produced herein did not allow the detection of the derivatized product.

Physicochemical Properties:

The presence of a weak acidic functional group, such as a carboxylic acid, on the [symbol] -986 was confirmed by an increase of its retention time on the HPLC C18 column when pH of the formate buffer was decreased from 7 to 3. This strongly suggests that a moiety possessing a pKa of about 4 or more is present in the [symbol] -986.

If a thiol or thioether functional group is present in the [symbol] -986, as suggested by the MS/MS data, it will affect the recovery of the [symbol] -986 from the 0-500 fraction and the cartilage. It is likely that only the free thiol portion of the [symbol] -986 can be extracted according to the present process as thiols tend to form disulfide (S═S) bonds with other sulfur containing molecules (such as proteins, peptide, amino acid) in solution. The formation of a disulfide adduct generally alters the physicochemical properties of the molecules containing thiol groups and affect their recovery by extraction. It is possible that a disulfide adduct of [symbol] -986 may not be isolated by direct extraction of the 0-500 fraction (20×). The formation of disulfide adducts of the [symbol] -986 can be minimized by treating solutions containing it with tributylphosphamide at pH 7 and room temperature for 15 minutes prior to extractions, especially those at pH 3 (SPE C 18 pH 3). Other disulfide bond-cleaving reagents, such as dithiothreitol and β-mercaptoethanol, can be used to minimize the formation of disulfide adducts of [symbol] -986.

The above processes for the recovery and the isolation of biological activities from shark cartilage can be adapted to any source of cartilage to extract fractions exhibiting desired biological activities.

This invention has been described hereinabove, with reference to specific embodiments. It is well within the ability of the skilled artisan to make modifications without departing from the above teachings. These modifications are within the scope of this invention as defined in the appended claims.

What is claimed is:

1. A process for obtaining a soluble biologically active component from cartilage comprising the steps of:
a) treating cartilage material with a quantity of organic solvent-containing solution to form a first mixture comprising a soluble component of cartilage; and
b) separating said first mixture to form a first liquid extract comprising said soluble component and a first mass of solids wherein said soluble component possesses at least anti-matrix metalloprotease or anti-proliferative activities.

2. The process of claim 1 further comprising the steps of:
a) removing a sufficient amount of liquid from said first liquid extract to form a substantially dry second mass of solids;
b) treating said second mass of solids with water to form a second mixture; and
c) separating said second mixture to form a final liquid extract and a third mass of solids, wherein said final liquid extract comprises said soluble component.

3. The process of claim 1 further comprising the step of:
removing substantially all of said organic solvent from said first liquid extract.

4. The process of claim 1 wherein said organic solvent-containing solution comprises one or more halogenated, ether, protic, aprotic, basic, acidic, polar, apolar, hydrophilic or hydrophobic solvents.

5. The process of claim 1 wherein said organic solvent-containing solution comprises one or more organic solvents selected from the group consisting of chloroform, dibromomethane, butyl chloride, dichloromethane, dimethoxymethane, tetrahydrofuran, diethyl ether, ethylene glycol dimethyl ether, ethylene glycol diethyl ether, diethylene glycol diethyl ether, triethylene glycol dimethyl ether, t-butyl ethyl ether, t-butyl methyl ether, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, anisole, benzyl alcohol, phenol, or glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide N-methylacetamide, N-methylformamide, trimethylamine (TMA), trifluoroacetic acid (TFA) and formic acid.

6. The process of claim 1 wherein said organic solvent-containing solution comprises one or more organic solvents selected from the group consisting of:
methanol, ethanol, acetonitrile, propanol, isopropanol, trimethylamine acetone and dimethylsulfoxide.

7. The process of claim 1 wherein said organic solvent-containing solution comprises a combination of organic solvents selected from the group consisting of:
methanol and acetonitrile, methanol and dimethylsulfoxide, ethanol and acetonitrile and ethanol and dimethylsulfoxide.

8. The process of claim 1 wherein said organic solvent-containing solution comprises a combination of water and an organic solvent selected from the group consisting of
methanol, propanol, isopropanol, ethanol, acetonitrile, trimethylamine, trifluoroacetic acid, formic acid and dimethylsulfoxide.

9. The process of claim 1 wherein said organic solvent-containing solution comprises an organic solvent present in an amount of about 0.1-100 v/v with respect to the total solution volume.

10. The process of claim 1 wherein said organic solvent is present in an amount of about 40-80 v/v with respect to the total solution volume.

11. The process of claim 1 wherein said organic solvent is an acidic or basic solvent and is present in an amount of at least about 10% v/v with respect to the total solution volume.

12. The process of claim 1 wherein said organic solvent is an acidic or basic solvent and is present in an amount of about 0.1-1% v/v with respect to the total solution volume.

13. The process of claim 1 wherein said organic solvent is either trimethylamine 10-40%, formic acid 0.1-1%, trifluoroacetic acid 0.1-1%, isopropanol 10-100%, acetonitrile 10-100% or ammonium hydroxide 0.1-1%, all percentages expressed in terms of v/v with respect total solution volume

14. The process of claim 1 wherein said first mixture is separated by one or more of centrifugation, filtration, dialysis and settling of solids followed by removal of a supernatant.

15. The process of claim 2 wherein said removing of liquid is done by one or more of evaporation, lyophilization, distillation azeotropic distillation, desiccation, liquid/liquid extraction, addition of organic solvent absorbent and rotovapping.

16. The process of claim 1 wherein said cartilage material is shark cartilage.

17. The process of claim 1 further comprising the step of:
homogenizing said cartilage material prior to, during, or after treatment of said cartilage material with organic solvent-containing solution.

18. The process of claim 17 wherein said homogenizing is done by one or more of physical and chemical means.

19. The process of claim 1 further comprising the steps of:
repeating steps a) and b), substituting the mass of solids for the cartilage material, to obtain at least one further liquid extract and combining said at least one further liquid extract with said first liquid extract.

20. A cartilage extract obtained from shark and from the process of claim 13.
